(19) United States
(12) Patent Application Publication
(10) Pub. No.: US 2011/0245183 A1
(43) Pub. Date: Oct. 6, 2011

(54) Topical Uses of Szeto-Schiller Peptides

(76) Inventor: Nicholas V. Perricone, Meriden, CT (US)

(21) Appl. No.: 12/982,155

(22) Filed: Dec. 30, 2010

Related U.S. Application Data
(63) Continuation-in-part of application No. 12/755,079, filed on Apr. 6, 2010.

(51) Int. Cl. A61K 8/64; A61Q 19/08; A61Q 19/00

(52) U.S. Cl. 514/18.8

(57) ABSTRACT

Provided are methods for improving the appearance aging or of peri-menopausal, menopausal, or post-menopausal skin comprising: applying to the skin tissue of a mammal in need of such regulation, a safe and effective amount of a composition comprising at least one Szeto-Schiller peptide and a dermatologically acceptable carrier. Compositions for use in the methods are also provided.

### CROSS-REFERENCE TO RELATED APPLICATIONS

This application is a continuation-in-part of co-pending U.S. patent application Ser. No. 12/755,079, filed Apr. 6, 2010, the content of which is incorporated herein by reference.

### FIELD OF THE INVENTION

The present invention relates to topical compositions to prevent and to treat skin aging and to improve the appearance of menopausal skin. More specifically, the present invention relates to topical compositions comprising Szeto-Schiller peptides to increase skin elasticity, reduce the appearance of fine lines, even out skin coloring, treat other visible signs of aging and to address severe skin dryness, dullness, loss of elasticity, or lack of radiance or to prevent or retard the appearance of exaggerated lines and wrinkles or spider vessels or red blotchiness, all visible conditions of peri-menopausal, menopausal or post-menopausal skin.

### BACKGROUND OF THE INVENTION

Superoxide anion (O₂•) is generated by mitochondria, oxidase compounds in the plasma membrane and cytoplasmic enzymes. In the presence of mitochondrial reactive oxygen species in the mitochondria, O₂•⁻ can be converted into hydrogen peroxide, which can diffuse out of mitochondria into cytoplasm. In the presences of high iron or nitric oxide, the diffused hydrogen peroxide can form the highly reactive hydroxide radical (OH•) or peroxynitrite (ONOO⁻).

Reactive oxygen species (ROS) such as hydroxide radical and peroxynitrite can damage cells by oxidizing membrane phospholipids, proteins and nucleic acids. Membrane lipids are major targets of ROS, and lipid peroxidation may lead to membrane dysfunction and alterations in cell permeability. Further, mitochondria are vulnerable to oxidative damage because they are constantly exposed to high levels of ROS. The mitochondrial DNA may undergo oxidation damage. Altered function of many metabolic enzymes in the mitochondrial matrix and electron transport chain are affected by protein oxidation and nitration.

These damaging effects of ROS are normally kept under control by endogenous antioxidant systems including glutathione, ascorbic acid, and enzymes such as superoxide dismutase, glutathione peroxidase and catalase. The antioxidant superoxidase dismutase (SOD) is particularly affected by oxidation overwhelmed by ROS, and the resulting oxidative damage can lead to cell death.

Szeto and Schiller have disclosed water soluble, highly polar, aromatic cationic peptides for significantly reducing the number of mitochondria undergoing mitochondrial permeability transition (U.S. Pat. No. 7,576,061 B2, incorporated herein by reference) and also for use in therapy as analgesics (U.S. Pat. No. 6,703,483; U.S. Pat. App. Pub. No. 2004/0176305 A1, incorporated herein by reference). The peptides have between three and twenty amino acids covalently joined by peptide bonds and alternating aromatic and basic amino acid residues.

Zhao et al., 279 J. Biol. Chem. 34682-34690 (2004), reported that Szeto-Schiller peptides targeted the inner mitochondrial membrane and potently reduced intracellular ROS and cell death caused by tBHP in neuronal cells. In addition, the Szeto-Schiller peptides decreased mitochondrial ROS production, inhibited mitochondrial permeability transition and swelling and prevented cytochrome c release induced by Ca²⁺ in mitochondria. ROS and MPT have been implicated in myocardial stunning, therefore, the inner mitochondrial membrane targeted antioxidants were implicated for treatment of aging and diseases associated with oxidative stress. More recent studies support potential use of Szeto-Schiller peptides for ischemia-reperfusion injury and neurodegenerative disorders. Szeto, 8 AAPS J. E277-E283 (2006).

Most recently, Szeto disclosed that three small, nitrated amino acid sequences carrying a 3+ charge at physiological pH were able to penetrate neuronal and endothelial cells of mice through intravenous administration. Ann. N.Y. Acad. Sci. 1147: 112-121 (2008). In isolated mitochondria, the addition of D-Arg-Dmt-Lys-Phe-NH2 (SS-31) and Phe-D-Arg-Phe-Lys-NH2 (SS-20) prevented MPP⁺-induced (1-methyl-4-phenylpyridium) inhibition of oxygen consumption, ATP production, and mitochondrial swelling. Moreover, mice studies showed that SS-31 and SS-20 may be effective in the treatment of Parkinson's Disease (PD) or Amyotrophic Lateral Sclerosis (ALS). The SS peptides were touted as having excellent pharmacokinetic properties and no adverse effects.

Cellular oxidative injury is implicated in a wide variety of clinical disorders in addition to ischemia-reperfusion injury and neurodegenerative diseases. In skin, reactive oxygen species such as singlet oxygen, the superoxide anion, and hydroxyl radicals, as well as other free radicals, are generated in normal metabolism, as well as through ultraviolet sun exposure, other forms of radiation, other environmental factors such as pollution or exposure to chemicals in the home or workplace, and the like. Free radicals activate chemical mediators that increase phospholipidase A2 resulting in the release of arachidonic acid from the cell membrane which is then oxidized by lipooxygenase and cyclooxygenase enzymes which produce leukotrines and prostaglandins, stimulating the inflammation cascade.

### SUMMARY OF THE INVENTION

The present invention provides topical compositions comprising a carrier and an effective amount of Szeto-Schiller peptide to treat skin aging, and to address many conditions experienced by women in the menopausal state.

Methods for improving the condition of and, preventing or treating aging and menopausal skin comprise applying a composition containing an effective amount of Szeto-Schiller peptide in a dermatologically acceptable carrier to skin.

More specifically, the present invention provides topical compositions and methods of applying compositions comprising Szeto-Schiller peptide for preventing and/or treating skin aging and to address severe skin dryness, dullness, loss of elasticity, or lack of radiance or to prevent or retard the appearance of exaggerated lines and wrinkles or spider vessels or red blotchiness, all visible conditions of peri-menopausal, menopausal, or post-menopausal skin.

### DETAILED DESCRIPTION OF THE INVENTION

Aging of skin cells often is associated with oxidative stress. It is believed that oxidative stress is caused by an imbalance between the production of reactive oxygen and a biological system's ability to neutralize the reactive intermediates. Oxidative damage occurs because of both intrinsic and extrinsic mechanisms. Together, intrinsic and extrinsic damage are the primary causes of skin aging. The skin uses a series of intrinsic antioxidants to protect itself from free radical damage. It has been demonstrated that topical antioxidant use can provide additional protection from oxidative damage, retard skin aging and improve skin appearance. The present invention recognizes this process and provides compositions and methods to minimize both prospective and existing aging conditions.

Free radicals interact with intracellular transcription factors that upregulate transcription factors such as activator protein 1 (AP-1), and nuclear transcription factor-kappa B (NF-kB). AP-1 is responsible for the production of metalloproteinases that break down existing collagen, contributing to wrinkle formation. NF-kB up-regulates the transcription of pro-inflammatory mediators including interleukin-1 (IL-1), IL-6, and IL-8, and tumor necrosis factor alpha (TNF-[alpha]). These pro-inflammatory mediators serve to further activate the transcription factors AP-1 and NF-kB, resulting in additional damage.

It is the sum of these events that is responsible for skin aging. The free radicals that are the most biologically significant include superoxide anion, peroxyl radical, pepoxy nitrite radical and hydroxyl radical. Damage caused by ROS lead, in the skin, to melanocytic overproduction of melanosomes and to weakened elastin and collagen. These changes also lead to a slower turnover of new skin cells. The cumulative effect is skin wrinkling, laxness, fragility, dull appearance, mottled brown pigmentation and distinct dark spots. DNA mutations caused by oxidative changes may also produce abnormal keratinocytes leading, in some cases, to malignancy.

Moreover, aging is often caused by the loss of estrogen or decline in oestrogen associated with menopause. Oestrogen receptors are most abundant around the face, genital area and lower limbs. Research supports the concept that oxygen stress contributes to menopause and that some of its physiopathological effects may be prevented and/or treated improving the antioxidant defense of menopausic and postmenopausic women. In particular, the concept of antioxidants that protect mitochondria against premature oxidative damage with loss of ATP synthesis and specialized cellular functions is supported. See e.g. Miguel, 42(3) Arch Gerontol Geriatr. 289-306 (2006). The present invention recognizes this process and provides a composition and method to minimize both prospective and existing skin conditions associated with loss of estrogen and oestrogen during menopause.

The present invention comprises topical compositions of Szeto-Schiller peptides (“SS peptide(s)”) to prevent or treat skin aging and address skin conditions associated with menopause. The compositions help address severe skin dryness, dullness, loss of elasticity, or lack of radiance exaggerated lines and wrinkles or spider vessels or red blotchiness. The treatments consist of topically applying SS peptides to the skin in a dermatologically acceptable carrier.

The term “skin” means the keratinous surfaces skin, hair and nails. The term “skin” when used herein is in the broad sense meaning the skin of the face, body, and neck as well as the lips.

### Szeto-Schiller Peptides

Szeto-Schiller peptides or “SS peptides” are small, aromatic-cationic, water soluble, highly polar peptides such as those disclosed in U.S. Pat. Nos. 6,703,483 (incorporated herein by reference) and 7,576,061 (incorporated herein by reference) that can readily penetrate cell membranes. The aromatic-cationic peptides include a minimum of three amino acids, and preferably include a minimum of four amino acids, covalently joined by peptide bonds. The maximum number of amino acids is about twenty amino acids covalently joined by peptide bonds. Optimally, the number of amino acids present in the SS peptides is four.

The amino acids of the aromatic-cationic SS peptides are any amino acid. Preferably, at least one amino group is at the alpha position relative to the carboxyl group. One or more chemical groups can be added to one or more of the 2′, 3′, 4′, 5′, or 6′ position of the aromatic ring of a phenylalanine or tyrosine residue, or the 4′, 5′, 6′, or 7′ position of the benzo ring of a tryptophan residue. Carboxyl groups, especially the terminal carboxyl group of a C-terminal amino acid, are preferably amidated with, for example, ammonia to form the C-terminal amide. Alternatively, the terminal carboxyl group of the C-terminal amino acid may be amidated with any primary or secondary amine.

In one embodiment, the amino acid at the N-terminus is phenylalanine or its derivative. Preferred derivatives of phenylalanine include 2′-methylphenylalanine (Mmp), 2′,6′-dimethylphenylalanine (Dmp), N,2′,6′-trimethylphenylalanine (Tmp), and 2′-hydroxy-6′-methylphenylalanine (Hmp).

The aromatic-cationic peptides have a minimum number of net positive charges at physiological pH in comparison to the total number of amino acid residues in the peptide.

The SS peptide having the formula Tyr-n-Arg-Phe-Lys-NH₂ (represented by the acronym: DALDA) is a recognized example, as are its functional analogs.

For purposes of the claims, the term “Szeto-Schiller peptide” is defined as the following:

(SEQ. ID. NO: 1)
Tyr-D-Arg-Phe-Lys-NH₂,
(SEQ. ID. NO: 2)
Dmt-D-Arg-Phe-Lys-NH₂,
(SEQ. ID. NO: 3)
Phe-D-Arg-Phe-Lys-NH₂,
(SEQ. ID. NO: 4)
D-Arg-Dmt-Lys-Phe-NH₂,
(SEQ. ID. NO: 5)
Dmp-D-Arg-Phe-Lys-NH₂,
(SEQ. ID. NO: 6)
D-Arg-Dmt-Phe-Lys-NH₂,
(SEQ. ID. NO: 7)
D-Arg-Phe-Lys-Dmt-NH₂,
(SEQ. ID. NO: 8)
D-Arg-Phe-Dmt-Lys-NH₂,
(SEQ. ID. NO: 9)
D-Arg-Lys-Dmt-Phe-NH₂,
(SEQ. ID. NO: 10)
D-Arg-Lys-Phe-Dmt-NH₂,
(SEQ. ID. NO: 11)
Phe-Lys-Dmt-D-Arg-NH₂,
(SEQ. ID. NO: 12)
Phe-Lys-D-Arg-Dmt-NH₂,
(SEQ. ID. NO: 13)
Phe-D-Arg-Dmt-Lys-NH₂,
(SEQ. ID. NO: 14)
Phe-D-Arg-Lys-Dmt-NH₂,
(SEQ. ID. NO: 15)
Phe-Dmt-D-Arg-Lys-NH₂,
(SEQ. ID. NO: 16)
Phe-Dmt-Lys-D-Arg-NH₂,
(SEQ. ID. NO: 17)
Lys-Phe-Dmt-D-Arg-NH₂,
(SEQ. ID. NO: 18)
Lys-Dmt-D-Arg-Phe-NH₂,
(SEQ. ID. NO: 19)
Lys-Dmt-Phe-D-Arg-NH₂,
(SEQ. ID. NO: 20)
Lys-D-Arg-Phe-Dmt-NH₂,
(SEQ. ID. NO: 21)
Lys-D-Arg-Dmt-Phe-NH₂,
(SEQ. ID. NO: 22)
D-Arg-Dmt-D-Arg-Phe-NH₂,
(SEQ. ID. NO: 23)
D-Arg-Dmt-D-Arg-Dmt-NH₂,
(SEQ. ID. NO: 24)
D-Arg-Dmt-D-Arg-Tyr-NH₂,
(SEQ. ID. NO: 25)
D-Arg-Dmt-D-Arg-Trp-NH₂,
(SEQ. ID. NO: 26)
Trp-D-Arg-Phe-Lys-NH₂,
(SEQ. ID. NO: 27)
Trp-D-Arg-Tyr-Lys-NH₂,
(SEQ. ID. NO: 28)
Trp-D-Arg-Trp-Lys-NH₂,
(SEQ. ID. NO: 29)
Trp-D-Arg-Dmt-Lys-NH₂,
(SEQ. ID. NO: 30)
D-Arg-Trp-Lys-Phe-NH₂,
(SEQ. ID. NO: 31)
D-Arg-Trp-Phe-Lys-NH₂,
(SEQ. ID. NO: 32)
D-Arg-Trp-Lys-Dmt-NH₂,
(SEQ. ID. NO: 33)
D-Arg-Trp-Dmt-Lys-NH₂,
(SEQ. ID. NO: 34)
D-Arg-Lys-Trp-Phe-NH₂,
(SEQ. ID. NO: 35)
D-Arg-Lys-Trp-Dmt-NH₂,
(SEQ. ID. NO: 36)
Cyclohexyl-D-Arg-Phe-Lys-NH₂,
Ala-D-Arg-Phe-Lys-NH₂,
(SEQ. ID. NO: 37)
Lys-D-Arg-Tyr-NH₂,
Phe-D-Arg-His,
D-Tyr-Trp-Lys-NH₂,
Trp-D-Lys-Tyr-Arg-NH₂,
(SEQ. ID. NO: 38)
Tyr-His-D-Gly-Met,
(SEQ. ID. NO: 39)
Phe-Arg-D-His-Asp,
(SEQ. ID. NO: 40)
Tyr-Arg-Phe-Lys-Glu-His-Trp-Arg,
(SEQ. ID. NO: 41)
Lys-Gln-Tyr-Arg-Phe-Trp,
(SEQ. ID. NO: 42)
Tyr-D-Arg-Phe-Lys-Glu-NH₂,
(SEQ. ID. NO: 43)
Met-Tyr-D-Lys-Phe-Arg,
(SEQ. ID. NO: 44)
D-His-Glu-Lys-Tyr-D-Phe-Arg,
(SEQ. ID. NO: 45)
Lys-D-Gln-Tyr-Arg-D-Phe-Trp-NH₂,
(SEQ. ID. NO: 46)
Phe-D-Arg-Lys-Trp-Tyr-D-Arg-His,
(SEQ. ID. NO: 47)
Gly-D-Phe-Lys-Tyr-His-D-Arg-Tyr-NH₂,
(SEQ. ID. NO: 48)
Val-D-Lys-His-Tyr-D-Phe-Ser-Tyr-Arg-NH₂,
(SEQ. ID. NO: 49)
Trp-Lys-Phe-D-Asp-Arg-Tyr-D-His-Lys,
(SEQ. ID. NO: 50)
Lys-Trp-D-Tyr-Arg-Asn-Phe-Tyr-D-His-NH₂,
(SEQ. ID. NO: 51)
Thr-Gly-Tyr-Arg-D-His-Phe-Trp-D-His-Lys,
(SEQ. ID. NO: 52)
Asp-D-Trp-Lys-Tyr-D-His-Phe-Arg-D-Gly-Lys-NH₂,
(SEQ. ID. NO: 53)
D-His-Lys-Tyr-D-Phe-Glu-D-Asp-D-His-D-Lys-Arg-Trp-
NH₂,
(SEQ. ID. NO: 54)
Ala-D-Phe-D-Arg-Tyr-Lys-D-Trp-His-D-Tyr-Gly-Phe,
(SEQ. ID. NO: 55)
Tyr-D-His-Phe-D-Arg-Asp-Lys-D-Arg-His-Trp-D-His-
Phe,
(SEQ. ID. NO: 56)
Phe-Phe-D-Tyr-Arg-Glu-Asp-D-Lys-Arg-D-Arg-His-Phe-
NH₂,
(SEQ. ID. NO: 57)
Phe-Tyr-Lys-D-Arg-Trp-His-D-Lys-D-Lys-Glu-Arg-D-
Tyr-Thr,
(SEQ. ID. NO: 58)
Tyr-Asp-D-Lys-Tyr-Phe-D-Lys-D-Arg-Phe-Pro-D-Tyr-
His-Lys,
(SEQ. ID. NO: 59)
Glu-Arg-D-Lys-Tyr-D-Val-Phe-D-His-Trp-Arg-D-Gly-
Tyr-Arg-D-Met-NH₂,
(SEQ. ID. NO: 60)
Arg-D-Leu-D-Tyr-Phe-Lys-Glu-D-Lys-Arg-D-Trp-Lys-D-
Phe-Tyr-D-Arg-Gly,
(SEQ. ID. NO: 61)
D-Glu-Asp-Lys-D-Arg-D-His-Phe-Phe-D-Val-Tyr-Arg-
Tyr-D-Tyr-Arg-His-Phe-NH₂,
(SEQ. ID. NO: 62)
Asp-Arg-D-Phe-Cys-Phe-D-Arg-D-Lys-Tyr-Arg-D-Tyr-
Trp-D-His-Tyr-D-Phe-Lys-Phe,
(SEQ. ID. NO: 63)
His-Tyr-D-Arg-Trp-Lys-Phe-D-Asp-Ala-Arg-Cys-D-Tyr-
His-Phe-D-Lys-Tyr-His-Ser-NH₂,
(SEQ. ID. NO: 64)
Gly-Ala-Lys-Phe-D-Lys-Glu-Arg-Tyr-His-D-Arg-D-Arg-
Asp-Tyr-Trp-D-His-Trp-His-D-Lys-Asp,
and
(SEQ. ID. NO: 65)
Thr-Tyr-Arg-D-Lys-Trp-Tyr-Glu-Asp-D-Lys-D-Arg-His-
Phe-D-Tyr-Gly-Val-Ile-D-His-Arg-Tyr-Lys-NH₂.

In a preferred embodiment, the SS peptide has the formula Tyr-D-Arg-Phe-Lys-NH₂ (SEQ. ID. NO: 1, DALDA, which is referred to herein as SS-01). DALDA has a net positive charge of three, contributed by the amino acids tyrosine, arginine, and lysine and has two aromatic groups contributed by the amino acids phenylalanine and tyrosine. The tyrosine of DALDA can be a modified derivative of tyrosine such as in 2′,6′ dimethyltyrosine to produce the compound having the formula 2′,6′-Dmt-D-Arg-Phe-Lys-NH₂ (SEQ. ID. NO: 2, i.e., Dmt-DALDA, which is referred to herein as SS-02).

In another preferred embodiment, the SS peptide has the formula Phe-D-Arg-Phe-Lys-NH₂ (SEQ. ID. NO: 3, i.e., Phe⁻¹-DALDA, which is referred to herein as SS-20). In a particularly preferred embodiment, the amino acid sequence of Dmt¹⁻DALDA (SS-02) is rearranged such that Dmt is not at the N-terminus, such as the formula D-Arg-2′,6′-Dmt-Lys-Phe-NH₂ (SEQ. ID. NO: 4, referred to in this specification as SS-31).

### Synthesis of Peptides

The peptides useful in the methods of the present invention may be chemically synthesized by any of the methods well known in the art. Suitable methods for synthesizing the protein include, for example those described by Stuart and Young in “Solid Phase Peptide Synthesis,” Second Edition, Pierce Chemical Company (1984), and in “Solid Phase Peptide Synthesis,” Methods Enzymol. 289, Academic Press, Inc, New York (1997).

### Compositions and Methods

A particular object of the present invention is to provide SS peptides to enhance skin penetration and transdermal absorption to improve the condition of the skin. The SS peptides are small and contain an amino acid sequence that allows them to freely penetrate cells, enabling the compounds of the invention to be effective as a topical application that can easily pass through the lipid bilayer of the cell membranes of epidermal and dermal cells. They are taken up into cells in an energy-independent nonsaturable manner. When having a D-amino acid in either the first or second position, they are resistant against aminopeptidase activity, and amidation of the C-terminus reduces hydrolysis from the C-terminus.

Topical compositions containing Szeto-Schiller peptides according to the present invention are intended to be topically applied to and absorbed by the skin tissue. While not wishing to be bound by any theory, it is believed that the SS peptides affect the appearance of aged, dull and dry skin cells because they have a sequence motif that targets them to mitochondria and is not dependent upon mitochondrial potential. They are localized to the inner mitochondrial membrane rather than in the matrix. By targeting and partitioning the inner mitochondrial membrane, the peptides are extremely potent in preventing oxidative cell death. The peptides can reduce intracellular ROS and cell death caused by tBHP.

Mitochondria permeability transition (MPT) may trigger tBHP induced apotosis. Peroxidation of cardiolipin induces the dissociation of cytochrome c from the inner mitochondrial membrane and subsequent release into the cytoplasm as a result of the opening of the MPT pore. Calcium overload can also lead to increase in mitochondrial ROS and opening of the MPT pore.

Antioxidant activity of SS peptides used the present invention can be attributed to the tyrosine or dimethyltyrosine (Dmt) residue. Tyrosine can scavenge oxyradicals forming relatively unreactive tyrosyl radicals, which can be followed by radical-radical coupling to give dityrosine, or react with superoxide to form tyrosine hyperoxide. Dimethyltyrosine is more effective than tyrosine in scavenging ROS. The specific location of the tyrosine or dimethyltyrosine residue is not as important as SS-31 was found to be as effective as SS-02 in scavenging H₂O₂ and inhibiting LDL oxidation. However, replacement of Dmt with phenylalanine (SS-20) eliminated the scavenging ability.

By reducing mitochondrial ROS, the scavenging SS-peptides may inhibit MPT, prevent mitochondrial swelling, and reduce cytochrome c release in response to Ca²⁺ overload. The non-scavenging peptides may not be as effective in prevention of mitochondrial swelling, and thus require higher concentrations.

After treatment for the recommended period of time, it is expected that decreased irritation and erythema of the skin will be observed, along with an increased skin elasticity and suppleness. Fine lines and wrinkles should be reduced and skin coloring should even out. The present invention thus is expected to prevent and treat skin aging, address skin dryness, dullness, loss of elasticity and lack of radiance. Particularly, treatments may be used to prevent or retard the appearance of spider vessels or red blotchiness associated with menopausal skin. In another embodiment, treatments may be used to prevent or retard exaggerated lines and wrinkles.

Only effective amounts of topical compositions containing SS peptide(s) are needed to achieve the aforementioned benefits and prevent typical menopausal and aging effects on the skin. Generally, topical application to skin tissue is accomplished in association with a dermatologically acceptable carrier, and particularly one in which the SS peptide is soluble per se or is effectively solubilized (e.g., as an emulsion or microemulsion). Where employed, the carrier is inert in the sense of not bringing about a deactivation or oxidation of the SS peptide, and in the sense of not bringing about any adverse effect on the skin areas to which it is applied.

In one preferred practice of the invention, one or more SS peptides is applied in admixture with the dermatologically acceptable carrier or vehicle (e.g., as a lotion, cream, ointment, soap, stick, or the like) so as to facilitate topical application and, in some cases, provide additional therapeutic effects as might be brought about, e.g., by moisturizing of the affected skin areas. More preferably, one or more peptides selected from the group consisting of Dmt-D-Arg-Phe-Lys-NH₂ (SEQ. ID. NO: 2), Phe-D-Arg-Phe-Lys-NH₂ (SEQ. ID. NO. 3) and D-Arg-Dmt-Lys-Phe-NH₂ (SEQ ID. NO: 4) is applied in admixture with the dermatologically acceptable carrier or vehicle. While the carrier for the topical composition can consist of a relatively simple solvent or dispersant such as water, it is generally preferred that the carrier comprise a composition more conducive to topical application, and particularly one which will form a film or layer on the skin to which it is applied so as to localize the application and provide some resistance to washing off by immersion in water or by perspiration and/or aid in the percutaneous delivery of the active agent(s). Many preparations are known in the art, and include lotions containing oils and/or alcohols and emollients vegetable oils, hydrocarbon oils and waxes, silicone oils, animal or marine fats or oils, glyceride derivatives, fatty acids or fatty acid esters, or alcohols or alcohol ethers, lecithin, lanolin and derivatives, polyhydric alcohols or esters, wax esters, sterols, phospholipids and the like, and generally also emulsifiers (nonionic, cationic or anionic), although some of the emollients inherently possess emulsifying properties. In the preferred embodiment, the carrier is lecithin.

As noted, these ingredients can be formulated into a cream, lotion, or gel, or a solid stick, by utilization of different proportions of the ingredients and/or by inclusion of thickening agents such as gums or other forms of hydrophilic colloids. One possible embodiment is a solution used to saturate a pad or wipe used to apply the compounds to affected areas; another is a cleanser; and others are lotions, creams, and gels, which are referred to herein as dermally or dermatologically acceptable carriers, and are formulated using conventional techniques known to those of ordinary skill in the art. The term “topical composition” as used herein shall mean the complete product including the SS peptide active ingredient, the carrier, and any adjuvants, thickeners, excipients, etc. as described herein which is applied to a person's skin.

The quantity of SS peptide active ingredient in the carrier may be varied or adjusted widely depending upon the particular application, the potency of the particular compound or the desired concentration. Generally, the quantity of SS peptide active ingredient will range between 10 ppm to 1000 ppm, and preferably 50 ppm to 500 ppm. Alternatively, the quantity of SS peptide active ingredient will range between 0.0001% to 0.1% by weight. In some applications, the quantity of SS peptide active ingredient will exceed 0.1% by weight. In different embodiments, the weight percentage of SS peptide may be in the range of 0.005%-0.0025%; 0.0025%-0.005%; 0.005%-0.01%; 0.01%-0.02%; 0.02%-0.03%; 0.03%-0.04%; 0.04%-0.05%; or 0.05%-0.1%. Generally, lower concentrations of SS peptide active ingredients in a carrier are suitable, depending upon the application regimen and the active and adjunct ingredients employed. Preferably, the peptide comprises up to 0.1% by weight of the composition. More preferably, the peptide is in the range from 0.1 ppm to 1000 ppm. Most preferably, the peptide is in the range from 1 to 10 ppm.

Generally in the practice of methods of the invention, the topical composition is topically applied to the skin areas, such as that of the face, at predetermined intervals often as a moisturizer, tinted foundation, cleanser, toner, lotion, cream, or gel, it generally being the case that gradual improvement is noted with each successive application. Insofar as has been determined based upon clinical studies to date, no adverse side effects are encountered. It is an advantage of the invention that compositions of the invention do not require a pharmaceutical prescription.

The topical composition of the invention can contain additional ingredients commonly found in skin care compositions and cosmetics, such as, for example, tinting agents, emollients, skin conditioning agents, emulsifying agents, humectants, preservatives, antioxidants, perfumes, chelating agents, etc., provided that they are physically and chemically compatible with other components of the composition. Preservatives include, but are not limited to, C₁-C₃ alkyl parabens and phenoxyenthanol, typically present in an amount ranging from about 0.1% to about 2.0% by weight percent, based on the total composition. Emollients, typically present in amounts ranging from about 0.01% to 5% of the total composition include, but are not limited to, fatty esters, fatty alcohols, mineral oils, polyether siloxane copolymers, and mixtures thereof. Humectants, typically present in amounts ranging from about 0.1% to about 5% by weight of the total composition include, but are not limited to, polyhydric alcohols such as glycerol, polyalkylene glycols (e.g., butylene glycol, propylene glycol, dipropylene glycol, polypropylene glycol, and polyethylene glycol) and derivatives thereof, alkylene polyols and their derivatives, sorbitol, hydroxy sorbitol, hexylene glycol, 1,3-dibutylene glycol, 1,2,6-hexanetriol, ethoxylated glycerol, propoxylated glycerol, and mixtures thereof. Emulsifiers, typically present in amounts from about 1% to about 10% by weight of the composition, include, but are not limited to, stearic acid, cetyl alcohol, stearyl alcohol, steareth 2, steareth 20, acrylates/C₁₀₋₃₀ alkyl acrylate crosspolymers, and mixtures thereof. Chelating agents, typically present in amounts ranging from about 0.01% to about 2% by weight, include, but are not limited to, ethylenediamine tetraacetic acid (EDTA) and derivatives and salts thereof, dihydroxyethyl glycine, tartaric acid, and mixtures thereof. Antioxidants, typically present in an amount ranging from about 0.02% to about 0.5% by weight of the composition, include, but are not limited to, butylated hydroxy toluene (BHT); vitamin C and/or vitamin C derivatives, such as fatty acid esters of ascorbic acid, particularly ascorbyl palmitate; butylated hydroanisole (BHA); phenyl-α-naphthylamine; hydroquinone; propyl gallate; nordihydroquiaretic acid; vitamin E and/or derivatives of vitamin E, including tocotrienol and/or tocotrienol derivatives; calcium pantothenates; green tea extracts; mixed polyphenols; and mixtures of any of these. Particularly preferred antioxidants are those that provide additional benefits to the skin such as ascorbyl palmitate.

Buffering agents are employed in many compositions. Preferably, the amount of buffering agent is one that results in compositions having a pH ranging from about 4.5 to about 8.5, more preferably from about 5.5 to about 8.5, most preferably from about 6.5 to about 8.0. Typical buffering agents are chemically and physically stable agents commonly found in cosmetics, and can include compounds that are also adjunct ingredients such as citric acid, malic acid, and glycolic acid buffers.

Some embodiments of this invention contain at least one other adjunct ingredient in addition to the SS peptide active ingredient. Adjunct ingredients include, but are not limited to one or more of: lipoic acid; α-hydroxy acids such as glycolic acid or lactic acid; ascorbic acid and its derivatives, especially fatty acid esters of ascorbic acid; or tocotrienols and tocotrienol derivatives and vitamin E compositions enriched with tocotrienols or tocotrienol derivatives. Additional ingredients and methods as disclosed in my U.S. Pat. Nos. 5,376,361; 5,409,693; 5,545,398; 5,554,647; 5,574,063; 5,643,586; 5,709,868; 5,879,690; 6,191,121; 6,296,861; 6,437,004; and 6,979,459, which are hereby incorporated by reference, may also be used.

A proposed composition in accordance with the present invention may comprise the following ingredients:

Ingredient

Aqua (water)
SS-31
lecithin
Tetrahexyldecyl Ascorbate
Phosphatidylcholine
Isopropyl Palmitate
Butylene Glycol
Glyceryl Stearate
PEG-100 Stearate
Cetearyl Alcohol
Oligopeptide-17
Ceteareth-20
Magnesium Aspartate
Dimethylaminoethanol (DMAE)
DHA
Thiotic Acid
fragrance

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

What is claimed is:

1. A method of improving the appearance of peri-menopausal, menopausal, or post-menopausal skin comprising: applying to the skin tissue of a mammal in need of such regulation, a safe and effective amount of a composition comprising at least one Szeto-Schiller peptide selected from the group consisting of: Dmt-D-Arg-Phe-Lys-NH₂ (SEQ. ID. NO: 2), Phe-D-Arg-Phe-Lys-NH₂ (SEQ. ID. NO. 3) and D-Arg-Dmt-Lys-Phe-NH₂ (SEQ ID. NO: 4); and a dermatologically acceptable carrier.

2. The method of claim 1 wherein the peptide is in the range from 0.1 ppm to 1000 ppm.

3. The method of claim 2 wherein the peptide is the range from 1 ppm to 10 ppm.

4. The method of claim 1 comprising up to 0.1% by weight of peptide.

5. The method of claim 1 wherein the carrier comprises lecithin.

6. The method of claim 1 wherein said composition further comprises one or more additional ingredients selected from the group consisting of: ascorbic acid and ascorbic acid derivatives; lipoic acid; α-hydroxy acids; and tocotrienols and tocotrienol derivatives and vitamin E compositions enriched with tocotrienols or tocotrienol derivatives.

7. The method of claim 1 wherein severe skin dryness, dullness, or lack of radiance are improved.

8. A method of preventing or retarding exaggerated lines and wrinkles of peri-menopausal, menopausal, or post-menopausal skin comprising: applying to the skin tissue of a mammal in need of such regulation, a safe and effective amount of a composition comprising at least one Szeto-Schiller peptide selected from the group consisting of: Dmt-D-Arg-Phe-Lys-NH₂ (SEQ. ID. NO: 2), Phe-D-Arg-Phe-Lys-NH₂ (SEQ. ID. NO. 3) and D-Arg-Dmt-Lys-Phe-NH₂ (SEQ ID. NO: 4); and a dermatologically acceptable carrier.

9. The method of claim 8 wherein the peptide is in the range from 0.1 ppm to 1000 ppm.

10. The method of claim 9 wherein the peptide is in the range from 1 ppm to 10 ppm.

11. The method of claim 8 comprising up to 0.1% by weight of peptide.

12. The method of claim 8 wherein the carrier comprises lecithin.

13. The method of claim 8 wherein said composition further comprises one or more additional ingredients selected from the group consisting of: ascorbic acid and ascorbic acid derivatives; lipoic acid; α-hydroxy acids; and tocotrienols and tocotrienol derivatives and vitamin E compositions enriched with tocotrienols or tocotrienol derivatives.

14. A method of preventing or retarding the appearance of spider vessels or red blotchiness in peri-menopausal, menopausal, or post-menopausal skin comprising: applying to the skin tissue of a mammal in need of such regulation, a safe and effective amount of a composition comprising at least one Szeto-Schiller peptide selected from the group consisting of: Dmt-D-Arg-Phe-Lys-NH₂ (SEQ. ID. NO: 2), Phe-D-Arg-Phe-Lys-NH₂ (SEQ. ID. NO. 3) and D-Arg-Dmt-Lys-Phe-NH₂ (SEQ ID. NO: 4); and a dermatologically acceptable carrier.

15. The method of claim 14 wherein the peptide is in the range from 0.1 ppm to 1000 ppm.

16. The method of claim 15 wherein the peptide is the range from 1 ppm to 10 ppm.

17. The method of claim 14 comprising up to 0.1% by weight of peptide.

18. The method of claim 14 wherein the carrier comprises lecithin.

19. The method of claim 14 wherein said composition further comprises one or more additional ingredients selected from the group consisting of: ascorbic acid and ascorbic acid derivatives; lipoic acid; α-hydroxy acids; and tocotrienols and tocotrienol derivatives and vitamin E compositions enriched with tocotrienols or tocotrienol derivatives.

20. A method of improving the appearance aging skin comprising: applying to the skin tissue of a mammal in need of such regulation, a safe and effective amount of a composition containing one or more peptides selected from the group consisting of Dmt-D-Arg-Phe-Lys-NH₂ (SEQ. ID. NO: 2), Phe-D-Arg-Phe-Lys-NH₂ (SEQ. ID. NO. 3) and D-Arg-Dmt-Lys-Phe-NH₂ (SEQ ID. NO: 4) in a dermatologically acceptable carrier.

21. The method of claim 20 wherein the amount peptide is in the range from 0.1 ppm to 1000 ppm.

22. The method of claim 21 wherein the amount of peptide is in the range from 1 ppm to 10 ppm.

23. The method of claim 20 comprising no more than 0.1% by weight of peptide.

24. The method of claim 20 wherein said composition further comprises one or more additional ingredients selected from the group consisting of: ascorbic acid and ascorbic acid derivatives; lipoic acid; α-hydroxy acids; and tocotrienols and tocotrienol derivatives and vitamin E compositions enriched with tocotrienols or tocotrienol derivatives.

25. The method of claim 20 wherein skin dryness, dullness, or lack of radiance are improved.

26. The method of claim 20 wherein exaggerated lines and wrinkles are prevented or retarded.

27. The method of claim 20 wherein the appearance of spider vessels or red blotchiness is prevented or retarded.

28. A topical composition to prevent and repair skin aging, comprising: an effective amount of at least one Szeto-Schiller peptide and a dermatologically acceptable carrier.

29. The topical composition of claim 28 further comprising one or more additional ingredients selected from the group consisting of: ascorbic acid and ascorbic acid derivatives; lipoic acid; α-hydroxy acids; and tocotrienols and tocotrienol derivatives and vitamin E compositions enriched with tocotrienols or tocotrienol derivatives.

30. The topical composition of claim 28 wherein the peptide is in the range from 0.1 ppm to 1000 ppm.

31. The topical composition of claim 30 wherein the peptide is in the range from 50 to 500 ppm.

32. The topical composition of claim 28 comprising up to 0.1% by weight of peptide.

33. The topical composition of claim 28 wherein the carrier comprises lecithin.

34. A method for the prevention and treatment of skin aging comprising: applying a composition containing Szeto-Schiller peptide selected from the group consisting of: Dmt-D-Arg-Phe-Lys-NH₂ (SEQ. ID. NO: 2), Phe-D-Arg-Phe-Lys-NH₂ (SEQ. ID. NO. 3) and D-Arg-Dmt-Lys-Phe-NH₂ (SEQ ID. NO: 4); and a dermatologically acceptable carrier to skin tissue.

35. The method of claim 34 wherein the peptide is in the range from 0.1 ppm to 1000 ppm.

36. The method of claim 35 wherein the peptide is in the range from 50 to 500 ppm.

37. The method of claim 34 comprising up to 0.1% by weight peptide.

38. The method of claim 34 wherein the carrier comprises lecithin.

39. The method of claim 34 wherein said composition further comprises one or more additional ingredients selected from the group consisting of: ascorbic acid and ascorbic acid derivatives; lipoic acid; α-hydroxy acids; and tocotrienols and tocotrienol derivatives and vitamin E compositions enriched with tocotrienols or tocotrienol derivatives.


SEQUENCE LISTING (65 sequences)

SEQ ID NO: 1 - protein, 4 aa, Artificial Sequence
  chemically sythesized or purified recombinant, mitochondria targeted aromatic-cationic peptide
YRFK

SEQ ID NO: 2 - protein, 4 aa, Artificial Sequence
  chemically sythesized or purified recombinant, mitochondria targeted aromatic-cationic peptide
YRFK

SEQ ID NO: 3 - protein, 4 aa, Artificial Sequence
  chemically sythesized or purified recombinant, mitochondria targeted aromatic-cationic peptide
FRFK

SEQ ID NO: 4 - protein, 4 aa, Artificial Sequence
  chemically sythesized or purified recombinant, mitochondria targeted aromatic-cationic peptide
RYKF

SEQ ID NO: 5 - protein, 4 aa, Artificial Sequence
  chemically sythesized or purified recombinant, mitochondria targeted aromatic-cationic peptide
FRFK

SEQ ID NO: 6 - protein, 4 aa, Artificial Sequence
  chemically sythesized or purified recombinant, mitochondria targeted aromatic-cationic peptide
RYFK

SEQ ID NO: 7 - protein, 4 aa, Artificial Sequence
  chemically sythesized or purified recombinant, mitochondria targeted aromatic-cationic peptide
RFKY

SEQ ID NO: 8 - protein, 4 aa, Artificial Sequence
  chemically sythesized or purified recombinant, mitochondria targeted aromatic-cationic peptide
RFYK

SEQ ID NO: 9 - protein, 4 aa, Artificial Sequence
  chemically sythesized or purified recombinant, mitochondria targeted aromatic-cationic peptide
RKYF

SEQ ID NO: 10 - protein, 4 aa, Artificial Sequence
  chemically sythesized or purified recombinant, mitochondria targeted aromatic-cationic peptide
RKFY

SEQ ID NO: 11 - protein, 4 aa, Artificial Sequence
  aromatic-cationic peptide
FKYR

SEQ ID NO: 12 - protein, 4 aa, Artificial Sequence
  chemically sythesized or purified recombinant, mitochondria targeted aromatic-cationic peptide
FKRY

SEQ ID NO: 13 - protein, 4 aa, Artificial Sequence
  chemically sythesized or purified recombinant, mitochondria targeted aromatic-cationic peptide
FRYK

SEQ ID NO: 14 - protein, 4 aa, Artificial Sequence
  chemically sythesized or purified recombinant, mitochondria targeted aromatic-cationic peptide
FRKY

SEQ ID NO: 15 - protein, 4 aa, Artificial Sequence
  chemically sythesized or purified recombinant, mitochondria targeted aromatic-cationic peptide
FYRK

SEQ ID NO: 16 - protein, 4 aa, Artificial Sequence
  chemically sythesized or purified recombinant, mitochondria targeted aromatic-cationic peptide
FYKR

SEQ ID NO: 17 - protein, 4 aa, Artificial Sequence
  chemically sythesized or purified recombinant, mitochondria targeted aromatic-cationic peptide
KFYR

SEQ ID NO: 18 - protein, 4 aa, Artificial Sequence
  chemically sythesized or purified recombinant, mitochondria targeted aromatic-cationic peptide
KYRF

SEQ ID NO: 19 - protein, 4 aa, Artificial Sequence
  chemically sythesized or purified recombinant, mitochondria targeted aromatic-cationic peptide
KYFR

SEQ ID NO: 20 - protein, 4 aa, Artificial Sequence
  chemically sythesized or purified recombinant, mitochondria targeted aromatic-cationic peptide
KRFY

SEQ ID NO: 21 - protein, 4 aa, Artificial Sequence
  chemically sythesized or purified recombinant, mitochondria targeted aromatic-cationic peptide
KRYF

SEQ ID NO: 22 - protein, 4 aa, Artificial Sequence
  chemically sythesized or purified recombinant, mitochondria targeted aromatic-cationic peptide
RYRF

SEQ ID NO: 23 - protein, 4 aa, Artificial Sequence
  aromatic-cationic peptide
RYRY

SEQ ID NO: 24 - protein, 4 aa, Artificial Sequence
  chemically sythesized or purified recombinant, mitochondria targeted aromatic-cationic peptide
RYRY

SEQ ID NO: 25 - protein, 4 aa, Artificial Sequence
  chemically sythesized or purified recombinant, mitochondria targeted aromatic-cationic peptide
RYRW

SEQ ID NO: 26 - protein, 4 aa, Artificial Sequence
  chemically sythesized or purified recombinant, mitochondria targeted aromatic-cationic peptide
WRFK

SEQ ID NO: 27 - protein, 4 aa, Artificial Sequence
  chemically sythesized or purified recombinant, mitochondria targeted aromatic-cationic peptide
WRYK

SEQ ID NO: 28 - protein, 4 aa, Artificial Sequence
  chemically sythesized or purified recombinant, mitochondria targeted aromatic-cationic peptide
WRWK

SEQ ID NO: 29 - protein, 4 aa, Artificial Sequence
  chemically sythesized or purified recombinant, mitochondria targeted aromatic-cationic peptide
WRYK

SEQ ID NO: 30 - protein, 4 aa, Artificial Sequence
  chemically sythesized or purified recombinant, mitochondria targeted aromatic-cationic peptide
RWKF

SEQ ID NO: 31 - protein, 4 aa, Artificial Sequence
  chemically sythesized or purified recombinant, mitochondria targeted aromatic-cationic peptide
RWFK

SEQ ID NO: 32 - protein, 4 aa, Artificial Sequence
  chemically sythesized or purified recombinant, mitochondria targeted aromatic-cationic peptide
RWKY

SEQ ID NO: 33 - protein, 4 aa, Artificial Sequence
  chemically sythesized or purified recombinant, mitochondria targeted aromatic-cationic peptide
RWYK

SEQ ID NO: 34 - protein, 4 aa, Artificial Sequence
  chemically sythesized or purified recombinant, mitochondria targeted aromatic-cationic peptide
RKWF

SEQ ID NO: 35 - protein, 4 aa, Artificial Sequence
  chemically sythesized or purified recombinant, mitochondria targeted aromatic-cationic peptide
RKWY

SEQ ID NO: 36 - protein, 4 aa, Artificial Sequence
  chemically sythesized or purified recombinant, mitochondria targeted aromatic-cationic peptide
ARFK

SEQ ID NO: 37 - protein, 4 aa, Artificial Sequence
  chemically sythesized or purified recombinant, mitochondria targeted aromatic-cationic peptide
WKYR

SEQ ID NO: 38 - protein, 4 aa, Artificial Sequence
  chemically sythesized or purified recombinant, mitochondria targeted aromatic-cationic peptide
YHGM

SEQ ID NO: 39 - protein, 4 aa, Artificial Sequence
  chemically sythesized or purified recombinant, mitochondria targeted aromatic-cationic peptide
FRHD

SEQ ID NO: 40 - protein, 6 aa, Artificial Sequence
  chemically sythesized or purified recombinant, mitochondria targeted aromatic-cationic peptide
YRFKEH

SEQ ID NO: 41 - protein, 6 aa, Artificial Sequence
  chemically sythesized or purified recombinant, mitochondria targeted aromatic-cationic peptide
KQYRFW

SEQ ID NO: 42 - protein, 5 aa, Artificial Sequence
  chemically sythesized or purified recombinant, mitochondria targeted aromatic-cationic peptide
YRFKE

SEQ ID NO: 43 - protein, 5 aa, Artificial Sequence
  chemically sythesized or purified recombinant, mitochondria targeted aromatic-cationic peptide
MYKFR

SEQ ID NO: 44 - protein, 6 aa, Artificial Sequence
  chemically sythesized or purified recombinant, mitochondria targeted aromatic-cationic peptide
HEKYFR

SEQ ID NO: 45 - protein, 6 aa, Artificial Sequence
  chemically sythesized or purified recombinant, mitochondria targeted aromatic-cationic peptide
KQYRFW

SEQ ID NO: 46 - protein, 7 aa, Artificial Sequence
  chemically sythesized or purified recombinant, mitochondria targeted aromatic-cationic peptide
FRKWYRH

SEQ ID NO: 47 - protein, 7 aa, Artificial Sequence
  chemically sythesized or purified recombinant, mitochondria targeted aromatic-cationic peptide
GFKYHRY

SEQ ID NO: 48 - protein, 8 aa, Artificial Sequence
  chemically sythesized or purified recombinant, mitochondria targeted aromatic-cationic peptide
VKHYFSYR

SEQ ID NO: 49 - protein, 8 aa, Artificial Sequence
  chemically sythesized or purified recombinant, mitochondria targeted aromatic-cationic peptide
WKFDRYHK

SEQ ID NO: 50 - protein, 8 aa, Artificial Sequence
  chemically sythesized or purified recombinant, mitochondria targeted aromatic-cationic peptide
KWYRNFYH

SEQ ID NO: 51 - protein, 9 aa, Artificial Sequence
  chemically sythesized or purified recombinant, mitochondria targeted aromatic-cationic peptide
TGYRHFWHK

SEQ ID NO: 52 - protein, 9 aa, Artificial Sequence
  chemically sythesized or purified recombinant, mitochondria targeted aromatic-cationic peptide
DWKYHFRGK

SEQ ID NO: 53 - protein, 10 aa, Artificial Sequence
  chemically sythesized or purified recombinant, mitochondria targeted aromatic-cationic peptide
HKYFEDHKRW

SEQ ID NO: 54 - protein, 10 aa, Artificial Sequence
  chemically sythesized or purified recombinant, mitochondria targeted aromatic-cationic peptide
AFRYKWHYGF

SEQ ID NO: 55 - protein, 11 aa, Artificial Sequence
  chemically sythesized or purified recombinant, mitochondria targeted aromatic-cationic peptide
YHFRDKRHWHF

SEQ ID NO: 56 - protein, 11 aa, Artificial Sequence
  chemically sythesized or purified recombinant, mitochondria targeted aromatic-cationic peptide
FFYREDKRRHF

SEQ ID NO: 57 - protein, 12 aa, Artificial Sequence
  chemically sythesized or purified recombinant, mitochondria targeted aromatic-cationic peptide
FYKRWHKKERYT

SEQ ID NO: 58 - protein, 12 aa, Artificial Sequence
  chemically sythesized or purified recombinant, mitochondria targeted aromatic-cationic peptide
YDKYFKRFPYHK

SEQ ID NO: 59 - protein, 13 aa, Artificial Sequence
  chemically sythesized or purified recombinant, mitochondria targeted aromatic-cationic peptide
ERKYVFHWRGYRM

SEQ ID NO: 60 - protein, 14 aa, Artificial Sequence
  chemically sythesized or purified recombinant, mitochondria targeted aromatic-cationic peptide
RLYFKEKRWKFYRG

SEQ ID NO: 61 - protein, 15 aa, Artificial Sequence
  chemically sythesized or purified recombinant, mitochondria targeted aromatic-cationic peptide
EDKRHFFVYRYYRHF

SEQ ID NO: 62 - protein, 16 aa, Artificial Sequence
  chemically sythesized or purified recombinant, mitochondria targeted aromatic-cationic peptide
DRFCFRKYRYWHYFKF

SEQ ID NO: 63 - protein, 17 aa, Artificial Sequence
  chemically sythesized or purified recombinant, mitochondria targeted aromatic-cationic peptide
HYRWKFDARCYHFKYHS

SEQ ID NO: 64 - protein, 19 aa, Artificial Sequence
  chemically sythesized or purified recombinant, mitochondria targeted aromatic-cationic peptide
GAKFKERYHRRDYWHWHKD

SEQ ID NO: 65 - protein, 20 aa, Artificial Sequence
  chemically sythesized or purified recombinant, mitochondria targeted aromatic-cationic peptide
TYRKWYEDKRHFYGVIHRYK